(19) United States
(12) Patent Application Publication
(10) Pub. No.: US 2020/0079733 A1
(43) Pub. Date: Mar. 12, 2020

(54) NOVEL IONIC LIQUIDS RESULTING FROM THE ASSOCIATION OF A SPECIFIC CATION AND A SPECIFIC ANION

(71) Applicant: COMMISSARIAT À L'ÉNERGIE ATOMIQUE ET AUX ÉNERGIES ALTERNATIVES, Paris (FR)

(72) Inventors: Stéphane Cadra, Saint Avertin (FR); Jonathan Szymczak, Quimper (FR); Matthieu Le Digabel, Monts (FR); Agnès Biller, Saint-Avertin (FR)

(21) Appl. No.: 16/496,132

(22) PCT Filed: Mar. 21, 2018
(86) PCT No.: PCT/FR2018/050682

§ 371 Date: Sep. 20, 2019

(30) Foreign Application Priority Data
Mar. 23, 2017 (FR) 17 52436

(51) Int. Cl. C07D 207/08; C07F 7/10; H01M 10/0568; H01M 10/0569; H01G 11/62; H01G 11/06

(52) U.S. Cl. CPC C07D 207/08; C07F 7/10; H01M 10/0568; H01G 11/52; H01G 11/62; H01G 11/06; H01M 2300/0045; H01M 10/0569

(57) ABSTRACT

Ionic liquids comprising the association of a cation have the following formula (I), in which —R1 is an acyclic hydrocarbonated group, n is a whole number between 0 and 3, and m is a whole number between 1 and 4, and an anion selected from a nitrate anion, a phosphate anion or an imidide anion.

### TECHNICAL FIELD

The present invention relates to novel ionic liquids resulting from the association between a specific cation and a specific anion, these ionic liquids having in particular proper conductivity properties, and more specifically a conductivity that can be higher than 1 mS/cm. The invention also relates to novel salts usable as ionic liquids in accordance with the invention or as intermediate products for designing ionic liquids in accordance with the invention.

A salt is obtained by associating an anionic compound (a fortiori, negatively charged) with a cationic compound (a fortiori, positively charged).

Among salts, ionic liquids are salts in the liquid state at room temperature (the melting point being lower than 20° C.), as opposed to conventional salts, as sodium chloride, which have a melting point close to 180° C., and these ionic liquids can be represented by the following general formula:

A⁺X⁻

in which:
A⁺ represents a generally organic cation; and
X⁻ represents an organic or inorganic anion.

The feature of ionic liquids in terms of state comes in particular from the morphological difference between the anion and the cation (for example, at the steric hindrance and geometry) poorly favourable for creating a crystalline form of the salt.

Further, ionic liquids have a low toxicity, a very low flammability, an electrochemical stability and an interesting ion conductivity.

Hence, ionic liquids are of a great interest in fields requiring the implementation of ion conducting solutions and can in particular be used as synthetic solvents, electrodeposition solutions or even electrolytes for energy storage devices, such as last-generation safe batteries, such as lithium-sulphur batteries, lithium-ion batteries or even redox flow batteries or even solar devices, such as dye-sensitised solar cells.

However, in the field of batteries, the limiting factor in these ionic liquids remains their high viscosity and their incompatibility toward some materials of electrodes, as is the case with graphite, which induces a limitation in battery performance in term of cycling, cycling commonly designating the number of charge/discharge cycles that can be made by a battery.

To overcome these drawbacks, some authors have worked on the modification of ionic liquids, for example, by adding particular functionalities to the cationic and/or anionic component for the purpose of improving the salt intrinsic properties and providing it with a particular functionality, for example, a lower viscosity.

In particular, Ferrari et al., in Journal of Power Sources, 194, 45-50, 2009 and Wu et al., in Electrochimica Acta, 184, 356-363, 2015 describe ionic liquids comprising pyrrolidinium cations the alkyl groups of which have been replaced with alkoxy groups, and the resulting ionic liquids can have a 12% reduction in viscosity with respect to their alkyl counterparts.

In view of what already exists, the authors of the present invention have developed novel ionic liquids which have in particular a significant conductivity (at least higher than 1 mS/cm) and a lesser viscosity compatible with a use of these ionic liquids as electrolytes and have also developed novel salts that can be used as ionic liquids in accordance with the invention or as intermediate salts for manufacturing these ionic liquids.

### DISCLOSURE OF THE INVENTION

These novel ionic liquids thus comprise the association of a cation having the following formula (I):

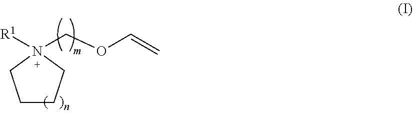

in which:
R¹ is an acyclic hydrocarbon group;
n is an integer ranging from 0 to 3;
m is an integer ranging from 1 to 4;

and an anion chosen from a nitrate anion, a phosphate anion or an imide anion.

It is intended that the cation of formula (I) and the abovementioned anions are associated so as to ensure electroneutrality of the resulting ionic liquid (in other words, an ionic liquid the positive charge(s) of said cation(s) of which balance the negative charge(s) of said anion(s)).

More explicitly, the cation of formula (I) can correspond, depending on the values of n, to one of the following formulae:
for n=0, the following formula (Ia):

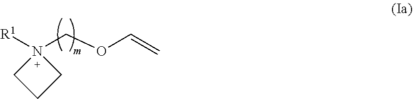

for n=1, the following formula (Ib):

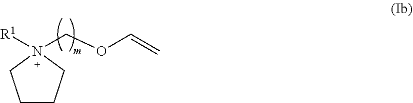

for n=2, the following formula (Ic):

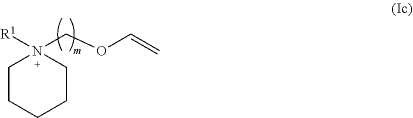

for n=3, the following formula (Id):

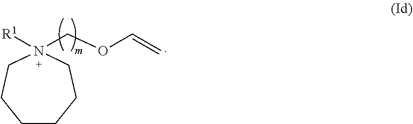

Advantageously, the ionic liquids of the invention are ionic liquids, in which the cation is a cation of the formula (I) with n being 1 (namely, in other words, a cation of formula (Ib)).

The group R¹ is an acyclic hydrocarbon group and, more specifically, it can be an acyclic, linear or branched hydrocarbon group, such as an alkyl group, including 1 to 4 carbon atoms. Further more specifically, the group R¹ can be a group of the formula —CₚH₂ₚ₊₁, with p being an integer ranging from 1 to 4, for example, a methyl group, an ethyl group, an n-propyl group, an isopropyl group, a n-butyl group, an isobutyl group, a tertiary butyl group.

By way of example, R¹ can be a methyl group.

By way of example, m can be 2.

As regards the anions:
when the anion is a nitrate anion, this has the formula NO₃⁻;
when the anion is a phosphate anion, this has the formula PO₄³⁻;
when the anion is an imide anion, this means conventionally that it includes an imide radical, the negative charge of which is carried by the nitrogen atom, which nitrogen atom is bonded to two carbonyl groups or two sulphonyl groups, and said imide radical can be represented by one of the following formulae (II) and (III):

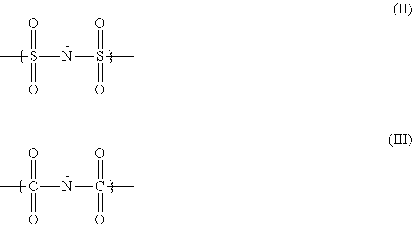

the braces indicating that the groups —SO₂— and —CO₂— are bonded to other groups.

Advantageously, the anion is an imide anion, the negative charge of which is carried by the nitrogen atom, which nitrogen atom is bonded to two sulphonyl groups, and such an anion can be represented by the following general formula (II′):

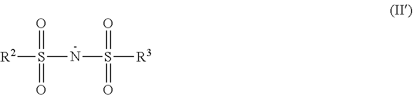

in which R² and R³ represent, independently of each other, a fluorine atom or a perfluorocarbon group.

More specifically, R² and R³ can both represent a fluorine atom or can both represent a perfluorocarbon group, for example, a perfluoromethyl group —CF₃.

Particular imide anions fulfilling these specificities are those of the following formulae (IV) and (V):

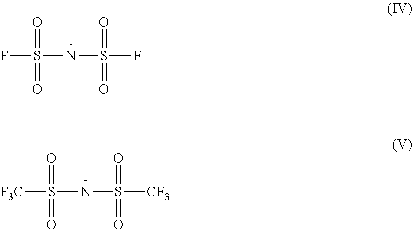

also known as bis(fluorosulphonyl)imide and bis(trifluoromethanesulphonyl)imide.

Ionic liquids in accordance with the invention are advantageously constituted of the association of a cation of the formula (Ib) and an anion having one of the abovementioned formulae (IV) and (V).

More specifically, for the cation of formula (Ib), R¹ can be a methyl group and m can be 2, in which case the cation has the following formula (IIb):

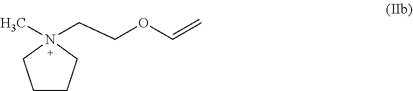

and this cation can be designated as N-(methyl)-(2-vinyloxyethyl)pyrrolidinium.

Ionic liquids in accordance with the invention are ionic liquids resulting from the association of a cation of the formula (IIb) and an anion of the formula (IV) or (V), which ionic liquids thus have the following respective formulae (VI) and (VII):

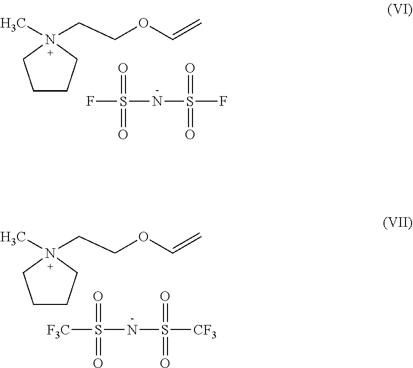

and these ionic liquids can be respectively designated as N-(methyl)-(2-vinyloxyethyl)pyrrolidinium bis(fluorosulphonyl)imide and N-(methyl)-(2-vinyloxyethyl)pyrrolidinium bis(trifluoromethanesulphonyl)imide.

The invention also relates to a novel family of salts, some of which make up ionic liquids as defined above or can be used as intermediate salts for manufacturing ionic liquids in accordance with the invention.

These salts in accordance with the invention comprise the association of at least one cation having the following formula (Ib):

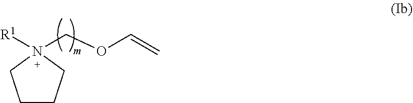

in which:
R¹ is an acyclic hydrocarbon group;
m is an integer ranging from 1 to 4;

and at least one anion Y.

The abovementioned listings regarding cations of the formula (I) for ionic liquids are also valid for these salts (in particular as regards R¹ and m).

It is intended that the cation(s) of the formula (Ib) and the anion(s) Y are associated so as to ensure electroneutrality of the resulting salt (in other words, a salt the positive charge(s) of said cation(s) of which balance the negative charge(s) of said anion(s)).

The anion Y can be an anion (in other words, the counter-ion associated with the cation of the formula (I)) chosen from halide anions (for example, chloride, bromide or iodide), a nitrate anion, a phosphate anion, imide anions. For example, the anion Y is a chloride type halide anion.

In the case where the anion Y is a nitrate anion, a phosphate anion or an imide anion, the resulting salts make up a specific class of ionic liquids in accordance with the invention.

The ionic liquids, in accordance with the invention, can be used alone or as a mixture with a salt different from those in accordance with the invention, which salt can be a lithium salt or a potassium salt, and said ionic liquids can be used as electrolytes, in particular, electrolytes for energy storage devices, such as lithium-ion batteries, lithium-sulphur batteries, redox flow batteries or even ultra-capacitors.

Thus, the invention also relates to an electrolyte comprising at least one ionic liquid as defined above.

The electrolyte can further comprise at least one lithium salt or at least one potassium salt.

According to a particular mode of the invention, the electrolyte can only consist of at least one ionic liquid as defined above or can further comprise at least one lithium salt or at least one potassium salt.

By way of examples of lithium salt, lithium hexafluorophosphate (LiPF₆), lithium bis(oxalatoborate), lithium tetrafluoroborate (LiBF₄), lithium bis(trifluoromethanesulphonyl)imide (known under the abbreviation LiTFSI), lithium bis(fluorosulphonyl)imide, lithium hexafluoroarsenate (LiAsF₆), lithium nitrate (LiNO₃) or even lithium perchlorate (LiCIO₄) can be mentioned.

By way of examples of potassium salt, potassium hexafluorophosphate (KPF₆), potassium tetrafluoroborate (KBF₄), potassium bis(trifluoromethanesulphonyl)imide, potassium bis(fluorosulphonyl)imide, potassium hexafluoroarsenate (KAsF₆), potassium nitrate (KNO₃) or even potassium perchlorate (KClO₄) can be mentioned.

The lithium salt or potassium salt can be included, in the electrolyte, at a concentration not exceeding 1.5 mole of salt per litre of ionic liquid.

The salts in accordance with the invention and ionic liquids in accordance with the invention can be prepared by any type of methods known to those skilled in the art, such as methods involving a substitution or an ion exchange.

By way of examples, when the salts in accordance with the invention include, as an anion, a halide anion, the preparation can consist in reacting a compound of the following formula (VIII):

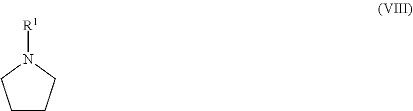

with R¹ being as defined above;

with a compound of the following formula (IX):

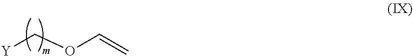

with m being such as defined above and Y corresponding to a halogen atom.

Without wishing to be bound by theory, the reaction of the compound of formula (VIII) with the compound of formula (IX) consists in a nucleophilic substitution generating a leaving group Y, which thus is a halide anion.

By way of examples, when the ionic liquids in accordance with the invention include, as an anion, an imide anion, a nitrate anion or a phosphate anion, the preparation can consist in an ion exchange between a salt consisting of the association of a cation of the abovementioned formula (I) with a halide anion (the salts for which the cation is of formula (Ib) being salts in accordance with the invention) and a salt consisting of the association of a cation, for example an alkaline cation (such as lithium, sodium or potassium) and an imide anion, a nitrate anion or a phosphate anion (this salt being designated hereinafter a secondary salt).

The ion exchange is typically performed in an aqueous medium and is characterised by the formation of two phases: a so-called organic phase comprising the salt comprising the association of a cation of formula (I) and an imide, nitrate or phosphate anion and an aqueous phase comprising the association of a halide anion and a cation, for example, an alkaline cation (from the secondary salt).

The aqueous phase is removed by settling and the organic phase undergoes a treatment that can involve an extraction operation using an organic solvent, and the new resulting organic phase can then be subjected to an active carbon filtration, an air vacuum drying and a zeolite dehydration. The dehydration can be extended until an amount of water in the ionic liquid lower than 50 ppm is obtained.

As already mentioned above, the ionic liquids of the invention as well as the above-defined electrolytes are of an important interest in the field of electrochemical storage namely due to, for example, their low flammability (which is an intrinsic property related to ionic liquids), a high conductivity (such as a conductivity higher than 2 mS/cm) and their melting temperature lower than or equal to −20° C.

In view of the abovementioned properties, the compositions comprising ionic liquids in accordance with the invention can be used as electrolytes, in particular, in an energy storage device, for example, a lithium secondary battery.

Thus, the invention also relates to a storage device comprising at least one cell comprising a positive electrode and a negative electrode separated from each other by a separator comprising an electrolyte in accordance with the invention. Further, the cell can be connected to a tank, which enables, via a pump, the electrolyte in accordance with the invention to be conveyed at the separator. The cell can also be connected to a charger to perform charging operations.

The positive electrode can be based on a carbon material and the negative electrode can be based on a lithium material, for example, a material based on metal oxide(s), such as Li₄Ti₅O₁₂.

The invention will now be described in reference to the examples provided below given by way of illustrating and non-limiting purposes.

### DETAILED DISCLOSURE OF PARTICULAR EMBODIMENTS

### Example 1

This example illustrates the preparation of a salt in accordance with the invention: N-(methyl)-(2-vinyloxyethyl)pyrrolidinium chloride of the following formula:

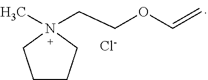

To do this, 25 g of 2-chlorovinyloxyethyl (0.234 mol) and 25 g of methylpyrrodiline (0.293 mol) are previously distilled and then diluted in 200 mL of acetonitrile. The mixture is kept under stirring for 120 hours at 30° C. The excess reagents and solvent are then evaporated under reduced pressure.

At the end of this evaporation, about 14 g of a yellow-orange coloured liquid (0.073 mol) are obtained, that is a yield of about 30%.

The liquid obtained is analysed by ¹H NMR and ¹³C NMR, the results of which are reported below.

¹H NMR (D₂O)=2.16 (br s, 4H); 3.02 (s, 3H); 3.67-3.49 (m, 6H); 4.35-4.17 (m, 4H); 6.2 (m, 1H).

¹³C NMR (D₂O)=21.1 (CH₂); 48.2 (CH₃); 62.2 (CH₂); 62.4 (CH₂); 65.3 (CH₂); 89.0 (CH₂); 150.1 (CH).

These results confirm that the product obtained is the salt having the formula defined above.

### Example 2

This example illustrates the preparation of an ionic liquid in accordance with the invention: N-(methyl)-(2-vinyloxyethyl)pyrrolidinium bis(fluorosulphonyl)imide of the following formula:

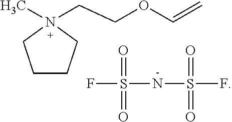

To do this, 19 g (0.099 mol) of the salt of example 1 and 22 g of potassium bis(fluorosulphonyl)imide (0.100 mL) are respectively dissolved in 100 mL of ultrapure water (having a resistivity of 18.2 mΩm⁻¹) to form two solutions (respectively, a solution comprising the salt of example 1 and a solution comprising potassium bis(fluorosulphonyl)imide). Both solutions are mixed at room temperature for 24 hours. An aqueous phase comprising potassium chloride and the excess reagents and an organic phase essentially comprising N-(methyl)-(2-vinyloxyethyl)pyrrolidinium bis(fluorosulphonyl)imide result from this mixture. The organic phase is recovered using dichloromethane and then transferred in a reparatory funnel, in order to be washed 5 times with 100 mL of ultrapure water. The organic phase is isolated and then the dichloromethane is removed by evaporation under reduced pressure, in order to obtain a raw ionic liquid. This ionic liquid is then diluted in ethyl acetate and then active carbon (13 g) is added. The mixture is placed under stirring for 96 hours at 35° C. The active carbon is then removed by filtration. The solution is then purified by adding about 25±5 g of alumina and then the resulting mixture is stirred for at least 5 hours at room temperature. The alumina is then removed by filtration and then the ionic liquid is recovered after removing ethyl acetate by vacuum evaporation (35° C., 1 mbar) for 72 hours.

At the end of this removal, about 24 g (that is 0.070 mol) of a pale yellow coloured liquid are obtained.

The liquid obtained is analysed by ¹H NMR and ¹³C NMR, the results of which are reported below.

¹H NMR (CDCl₃)=2.10 (br s, 4H); 2.90 (s, 3H); 3.55-3.39 (m, 6H); 4.22-3.94 (m, 4H); 6.32 (m, 1H).

¹³C NMR (CDCl₃)=21.0 (CH₂); 48.4 (CH₃); 62.1 (CH₂); 62.3 (CH₂); 65.4 (CH₂); 88.9 (CH₂); 150.2 (CH).

These results confirm that the product obtained is the ionic liquid having the formula defined above.

### Example 3

This example illustrates the preparation of an ionic liquid in accordance with the invention: N-(methyl)-(2-vinyloxyethyl)pyrrolidinium bis(trifluoromethanesulphonyl)imide of the following formula:

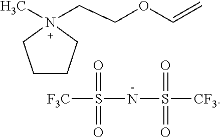

The synthesis protocol is similar to that described in the framework of example 2, except that 14 g of N-(methyl)-(2-vinyloxyethyl)pyrrolidinium chloride (0.073 mol) have been used and 21 g of lithium bis(trifluoromethanesulphonyl)imide (0.073 mol) have been used in place of 22 g of potassium bis(fluorosulphonyl)imide.

22 g of a pale yellow ionic liquid are obtained.

The liquid obtained is analysed by ¹H NMR and ¹³C NMR, the results of which are reported below.

¹H NMR (CDCl₃)=2.17 (br s, 4H); 3.02 (s, 3H); 3.55-3.35 (m, 6H); 4.55-4.02 (m, 4H); 6.20 (m, 1H)

¹³C NMR (CDCl₃)=21.1 (CH₂); 48.4 (CH₃); 62.3 (CH₂); 62.5 (CH₂); 65.54 (CH₂); 89.0 (CH₂); 120 (q, CF₃); 150.3 (CH).

These results confirm that the product obtained is the ionic liquid having the formula defined above.

### Example 4

In this example, the ionic liquids obtained in examples 2 and 3 are characterised in terms of conductivity (mS/cm) at 25° C., viscosity (mPa·s) at 25° C. and crystallisation temperature (° C.).

The results are reported in the table below.

| | | | Crystallisation |
|---|---|---|---|
| Ionic | Conductivity | Viscosity | temperature |
| liquid | (mS/cm) | (mPa · s) | (° C.) |
| Example 2 | 4.7 | 79.2 | −65 |
| Example 3 | 2.5 | 100.8 | −67 |

The ionic liquids have conductivity values higher than 2 mS/cm and melting temperatures lower than −20° C. Hence, these ionic liquids are of a very particular interest for use in electrochemical storage systems.

What is claimed is:

1.-16. (canceled)

17. An ionic liquid comprising the association of a cation having the following formula (I): 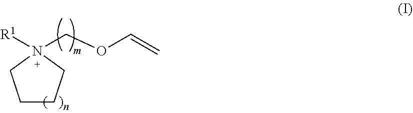
in which:
R¹ is an acyclic hydrocarbon group;
n is an integer ranging from 0 to 3;
m is an integer ranging from 1 to 4;
and an anion chosen from a nitrate anion, a phosphate anion or an imide anion.

18. The ionic liquid according to claim 17, wherein the cation of formula (I) has the following specific formula (Ib):

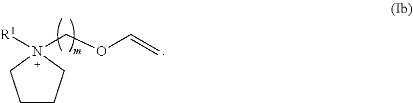

19. The ionic liquid according to claim 17, wherein R¹ is an alkyl group comprising from 1 to 4 carbon atoms.

20. The ionic liquid according to claim 17 wherein m is 2.

21. The ionic liquid according to claim 17, wherein the anion is an imide anion.

22. The ionic liquid according to claim 17, wherein the anion is an imide anion having the following formula (II′): 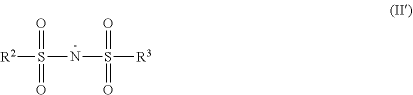
in which R² and R³ represent, independently of each other, a fluorine atom or a perfluorocarbon group.

23. The ionic liquid according to claim 17, wherein the anion is an imide anion which has one of the following formulae (IV) and (V):

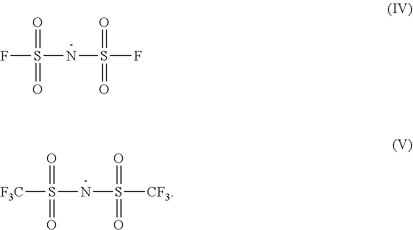

24. The ionic liquid according to claim 17, which has one of the following formulae (VI) and (VII):

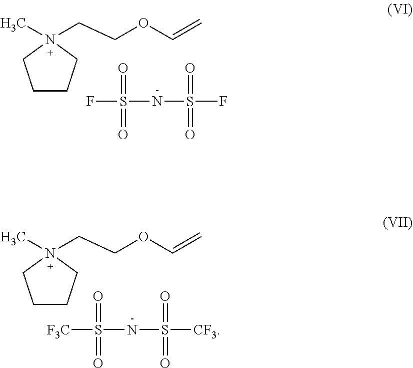

25. A salt comprising the association of at least one cation having the following formula (Ib): 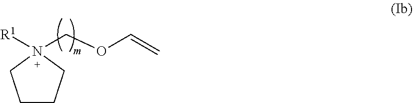
in which:
R¹ is an acyclic hydrocarbon group;
m is an integer ranging from 1 to 4;
and at least one anion Y.

26. The salt according to claim 25, wherein the anion Y is chosen from halide anions, a nitrate anion, a phosphate anion, imide anions.

27. The salt according to claim 25, wherein the anion Y is a chloride type halide anion.

28. An electrolyte comprising at least one ionic liquid as defined in claim 17.

29. The electrolyte according to claim 28, further comprising at least one lithium salt or at least one potassium salt.

30. The electrolyte according to claim 29, wherein the lithium salt is chosen from lithium hexafluorophosphate (LiPF₆), lithium bis(oxalatoborate), lithium tetrafluoroborate (LiBF₄), lithium bis(trifluoromethanesulphonyl)imide (known under the abbreviation LiTFSI), lithium bis(fluorosulphonyl)imide, lithium hexafluoroarsenate (LiAsF₆), lithium nitrate (LiNO₃) or even lithium perchlorate (LiCIO₄).

31. The electrolyte according to claim 29, wherein the potassium salt is chosen from potassium hexafluorophosphate (KPF₆), potassium tetrafluoroborate (KBF₄), potassium bis(trifluoromethanesulphonyl)imide, potassium bis(fluorosulphonyl)imide, potassium hexafluoroarsenate (KAsF₆), potassium nitrate (KNO₃) or even potassium perchlorate (KClO₄).

32. An energy storage device comprising at least one cell comprising a positive electrode and a negative electrode which are separated from each other by a separator comprising an electrolyte as defined in claim 28.
